(19) United States
(12) Patent Application Publication
(10) Pub. No.: US 2008/0311085 A1
(43) Pub. Date: Dec. 18, 2008

(54) Method for treating an animal having damaged tissue structure

(76) Inventors: Patrick J. Casey, Kumeu (NL); Kerri Fry, Kumeu (NL)

Correspondence Address: PATRICK R. SCANLON;PRETI FLAHERTY BELIVEAU & PACHIOS LLP, ONE CITY CENTER, PORTLAND, ME, 04112-9546, US

(21) Appl. No.: 12/005,153

(22) Filed: Dec. 21, 2007

Related U.S. Application Data
(60) Provisional application No. 60/876,373, filed on Dec. 21, 2006.

(51) Int. Cl. A61K 35/12; A61P 19/04

(52) U.S. Cl. 424/93.7

(57) ABSTRACT

A method for treating an animal having damaged tissue structure includes harvesting a tissue sample from a subject harvesting a tissue sample from the animal, growing tendon-like cells from the tissue sample, and transplanting the tendon-like cells into the damaged tissue structure. Growing the tendon-like cells from the tissue sample can be accomplished by breaking the tissue sample into fragments, placing the fragments into a culture vessel, inducing at least some of the fragments to adhere to the culture vessel, and supplying the fragments with nutrients so that tendon-like cells contained therein divide and grow.

### CROSS REFERENCES TO RELATED APPLICATIONS

This application claims the benefit of U.S. Provisional Application No. 60/876,373, filed Dec. 21, 2006.

### BACKGROUND OF THE INVENTION

Many animals, and horses in particular, are highly susceptible to tendon and ligament injuries because of the large amount of force exerted on these structures. Treatment options for tendon injuries are limited and often ineffective. Tendon injuries such as strains, tears or ruptures are particularly difficult to treat in horses and tend to heal poorly. Ligaments can also be sprained or ruptured. Horses often never fully recover from severe tendon or ligament injuries, and there can be a high rate of recurrence for such injuries.

Accordingly, it would be desirable to develop improved methodologies for treating horses and other animals having tendon or ligament injuries.

### SUMMARY OF THE INVENTION

The above-mentioned need is met by the present invention, one embodiment of which includes a method for treating an animal having damaged tissue structure that includes harvesting a tissue sample from the animal, growing tendon-like cells from the tissue sample, and transplanting the tendon-like cells into the damaged tissue structure. Growing the tendon-like cells from the tissue sample can comprise breaking the tissue sample into fragments, placing the fragments into a culture vessel, inducing at least some of the fragments to adhere to the culture vessel, and supplying the fragments with nutrients so that tendon-like cells contained therein divide and grow.

The present invention and its advantages over the prior art will be more readily understood upon reading the following detailed description.

### DETAILED DESCRIPTION OF THE INVENTION

The present invention generally relates to an in vitro method for growing tendon-like cells from a tissue sample taken from a mammal or other organism having damaged tissue structure, and transplanting the grown cells back into the same mammal or organism to assist in repairing the damaged tissue structure. The method applies in particular to repairing dense connective tissue such as ligaments and tendons. As used herein, the term “tendon-like cells” refers to tendon cells, ligament cells or any other dense connective tissue cells. In general, the method comprises harvesting a tissue sample from a patient and using the tissue sample to grow adult tendon-like cells under specific culture conditions. For example, cells originating from an equine nucal ligament can form tendon-like structures in vitro. While the present invention is particularly applicable to mammals such as horses, it should be noted that the present invention is not limited to mammalian cells.

In one embodiment, the method is performed on an animal, such as a horse, having a ruptured tendon (i.e., a discrete core lesion). The first step is to harvest one or more tissue samples from the injured animal using a minimally invasive biopsy technique. The samples can be taken from the ruptured tendon itself, but to ensure there is no further damage caused to the injured tendon, the samples are preferably taken from a surrogate tissue source. The surrogate tissue source should be a tissue that is similar to the damaged tissue and will form similar cell structures in vitro. For instance, two samples can be taken from the animal's nuchal ligament using a spring-loaded biopsy instrument under local anesthesia.

The harvested samples are transported to the laboratory at 4 degrees Celsius for further processing. Cellular growth is stimulated by breaking the tissue samples down into fragments. Fragmentation may be accomplished with any suitable technique, including but not limited to dissection, chemical digestion, and physical digestion. The fragments are placed in a culture vessel and induced to attach or adhere to the vessel, which will allow cells to grow and divide. The attached tissue fragments are then supplied with nutrients (for example, but not limited to, immersion in specific cell culture media under predetermined conditions) to induce the division/multiplication of adult tenocytes.

In one embodiment, the cells are placed in a modified “hanging drop” culture system wherein the fragments are placed into a culture flask with only enough media to keep tissue moist, not suspended. This flask is then flipped over (or partially tipped) and a small amount of media is added to the bottom to keep the tissue fragments humidified. With the flask inverted, the fragments are stressed by gravity but tend to remain in contact with the flask surface due to the moisture from the media.

Cells are grown in the laboratory until there are enough cells to transplant back into the core lesion, at which point cells can be harvested. For example, tenocytes are harvested using a Trypsin/EDTA incubation technique after a sufficient passage of time, such as 14 days. Between 10-20 million cells can be harvested. Of considerable interest is that the tenocytes grown in this manner show strong linear cellular alignment in a tendon like fashion.

The harvested tenocytes are injected back into the core lesion of the same injured animal, thus providing cells of the correct type that avoid rejection. The injected cells will aid in the repair of the injured tendon. Typically, the cells are trypsonised prior to implantation, loaded into a syringe and injected back into the same mammal that the samples came from (autotransplantation). The reintroduction procedure can be done with the animal standing, using sedation and a ring block.

The procedure has been tested on a number of injured horses. In each instance, tenocytes have been introduced into the core lesion with no untoward sequela. Fourteen days after transplantation an ultrasound examination was conducted on each horse and all core lesions showed significant infilling. Also no overgrowth of cells was evident outside the tendon fracture margins, which suggests that the cellular growth is limited by spatial compaction.

While specific embodiments of the present invention have been described, it should be noted that various modifications thereto can be made without departing from the spirit and scope of the invention as defined in the appended claims.

What is claimed is:

1. A method for treating an animal having damaged tissue structure, said method comprising:
harvesting a tissue sample from said animal;
growing tendon-like cells from said tissue sample; and
transplanting said tendon-like cells into said damaged tissue structure.

2. The method of claim 1 wherein said damaged tissue structure is a tendon.

3. The method of claim 1 wherein said damaged tissue structure is a ligament.

4. The method of claim 1 wherein said tissue sample is harvested from said animal's nuchal ligament.

5. The method of claim 1 wherein growing tendon-like cells from said tissue sample comprises:
breaking said tissue sample into fragments;
placing said fragments into a culture vessel;
inducing at least some of said fragments to adhere to said culture vessel; and
supplying said fragments with nutrients so that tendon-like cells contained therein divide and grow.

6. The method of claim 5 wherein inducing at least some of said fragments to adhere to said culture vessel includes:
placing said fragments into said culture vessel;
adding enough media to keep said fragments moist but not suspended;
flipping said culture vessel over; and
incubating said fragments.

7. The method of claim 5 wherein breaking the tissue sample into fragments includes dissection.

8. The method of claim 5 wherein breaking the tissue sample into fragments includes chemical digestion.

9. The method of claim 5 wherein breaking the tissue sample into fragments includes physical digestion.

10. The method of claim 5 further comprising removing tendon-like cells from said culture vessel after a sufficient number of cells have grown.

11. The method of claim 10 wherein cells are removed via Trypsin/EDTA incubation.
